(19) United States
(12) Patent Application Publication
(10) Pub. No.: US 2004/0260082 A1
(43) Pub. Date: Dec. 23, 2004

(54) Extraction of polysaccharides from vegetable and microbial material

(76) Inventors: Wim Van Der Wilden, Rotterdam (NL); Ingrid Karin Haaksman, Amersfoort (NL); Peter Frank Ekhart, Amsterdam (NL); Jan Matthijs Jetten, Zeist (NL)

Correspondence Address: YOUNG & THOMPSON, 745 SOUTH 23RD STREET, 2ND FLOOR, ARLINGTON, VA, 22202, US

(21) Appl. No.: 10/484,265

(22) PCT Filed: Jul. 17, 2002
(86) PCT No.: PCT/NL02/00482

(30) Foreign Application Priority Data
Jul. 17, 2001 (NL) 1018568

(51) Int. Cl. C12P 19/04; C08B 37/00

(52) U.S. Cl. 536/123.12; 536/124; 435/101

(57) ABSTRACT

Useful polysaccharides, such as β-1,3-glucans, from a biological raw material can be solubilised and/or isolated by treating the raw material with an oxidising agent that leads to oxidation of primary hydroxyl groups in the glucan. The oxidising agent is preferably a catalytic amount of a nitroxyl compound in the presence of a re-oxidising agent such as hypochlorite or an oxidative enzyme with oxygen or hydrogen peroxide. The polysaccharide retains its useful properties during this treatment and is, moreover, more readily available. If desired, protein material from the raw material can also be utilised.

Extraction of polysaccharides from vegetable and microbial material The invention relates to a process for the treatment of biological material, such as yeast cells and residues and extracts from vegetable material, with a view to the extraction and utilisation of polysaccharides and derivatives thereof.

The cell wall of various varieties of yeast such as baker's yeast (Saccharomyces cerevisiae) consists predominantly of polysaccharides (80-90% of the solids). These polysaccharides are mostly glucans and mannans and a small amount of chitin. The inner layer contains β-1,3- and β-1,6-glucans and the outer layer contains mannoproteins, which, in turn, are often covalently bonded to the interior glucan layer. Yeast cell walls also contain varying amounts of proteins, fats and inorganic phosphates. In industrial yeast cell wall preparations the protein content is frequently higher (15-30%) and the polysaccharide content correspondingly lower.

The β-glucans and mannoproteins have useful properties. The β-glucans, which consist of a chain of β-1,3-linked glucopyranosyl units with side β-1,6-linked gluco-pyranosyl units, strengthen the human immune system. This leads to tumour-suppressant, anti-bacterial, anti-viral, coagulation-inhibiting and wound-healing actions (Bohn, J. A. and BeMiller, J. M. (1995) Carbohydrate Polymers, 28, 3-14). The mannoproteins are found to be usable as an emulsifier (Cameron, D. R. et al. (1998) Appl. Environm. Microbiol. 54, 1420-1425); they can only be extracted by enzymatic degradation of the glucans and thus with no utilisation thereof.

Vegetable residual material, such as sugar beet pulp, sugar cane residues and brewer's grains, often contains appreciable amounts of valuable polysaccharides, such as β-glucans, arabinoxylans and cellulose, which could be suitable as dietary fibres for humans and animals, prebiotics, fat substitutes, thickeners, emulsifiers, moistening agents and the like.

Although the yeast cell residues and other microbial and vegetable residual materials thus constitute a potentially valuable raw material, the utilisation of this material has hardly been developed to date. An important reason is that the methods available up to now for the extraction of the polysaccharides and proteins, such as autoclave extraction (see Torabizadeh et al. (1996) Lebensm.-Wiss. u.-Technol. 29, 734), are too expensive.

It has been proposed by Ohno et al. (Carbohydrate Res. 316 (1999) 161-172) to solubilise β-(1,3)-glucans from yeast cell walls by oxidation with sodium hypochlorite and extraction of the insoluble fraction of the oxidation product with dimethyl sulphoxide. The oxidation is carried out in 0.1 M NaOH. With this method at most 14% (m/m) of the dried yeast cells is finally isolated as a β-(1,3)-glucan fraction. The product has an average molecular weight of 10⁶ Da, with a wide spread in molecular weight, and contains hardly any anionic groups. A large portion of the polysaccharide material has apparently been converted to non-extractable degradation products. Further disadvantages of this approach are that undesired functional groups, such as ketone functional groups and chlorine atoms, are incorporated and that undesired solvents such as dimethyl sulphoxide are required.

It has now been found that polysaccharides can be efficiently solubilised from a biological raw material and, if desired, isolated by oxidation with agents and under conditions such that primary hydroxyl groups are oxidised exclusively or virtually exclusively. It has furthermore been found that the polysaccharides oxidised and isolated in this way have retained their useful biological properties and, as a result of their increased solubility, find wider application than the untreated polysaccharides. Especially in the case of more extensive oxidation, products are obtained which are particularly suitable as an emulsifier, binder or thickener, for example in cosmetics or in foods.

The advantage of the process according to the invention compared with the process of Ohno et al. (see above) in the case of yeast material is that the β-1,3-glucans themselves are oxidised and specifically are oxidised in a controlled manner, valuable, well-defined derivatives being obtained. With the process according to Ohno et al. presumably mainly other materials, such as proteins, mannans and β-1,6-glucans are oxidised and further degraded, whilst a portion of the β-1,3-glucans is also lost as a result of uncontrolled oxidation and degradation. Moreover, according to the invention a much larger proportion of the starting material is usefully used: specifically approximately 80% instead of at most 14% according to Ohno et al. Furthermore, the product from the process according to the invention has a depleted content of β-1,6-glucan derivatives, which are usually less desired.

Here polysaccharides are understood to be saccharides having on average more than 10 monomer units, as well as derivatives of polysaccharides, proteoglycans, glyco-proteins and the like. The polysaccharides concerned are in particular polysaccharides which beforehand are insoluble or poorly soluble in water (less than 2 g per 100 g). The chain length (degree of polymerisation, DP) can be as high as, for example, 10,000 or more (molecular weight approximately 2,500,000) and is in particular 20-3,000 and more particularly 40-1000. The polysaccharides are present in the biological raw material in amounts of 1-75% (m/m), in particular 2-40% (m/m) (dry weight), the other material usually comprising protein.

With the process according to the invention no prior separation between poly-saccharides and other biological material, in particular proteins, is needed. The biological raw material generally contains this other biological material in an amount of at least 8% (m/m), usually in an amount of more than 10%. If desired, pretreatments such as denaturing or other partial protein degradation, fat removal, digestion, and the like can be carried out. Digestion, can, for example, be effected by swelling in alkali (pH 10-13), as a result of which the material becomes more readily accessible to the oxidative reagents.

The oxidation of primary hydroxyl groups in polysaccharides is known per se. This oxidation has been described for, inter alia, starch, cellulose and other glucans and can, for example, be carried out with nitrogen oxides (NO₂/N₂O₄ or nitrite/nitrate; see Netherlands Patent Application 9301172) and especially with nitroxyl compounds in the presence of a re-oxidising agent such as hypochlorite, peracetic acid or persulphuric acid (see WO 95/07303 and WO. 99/57158). The re-oxidising agent for the nitroxyl compounds can also be hydrogen peroxide or oxygen, in which case, for example, an oxidative enzyme such as a peroxidase, a laccase or another phenol oxidase, or a metal complex is present; see WO 00/50388 and WO 00/50621). With these oxidation methods an aldehyde can be formed in the first instance, which is then converted to a carboxylic acid.

The nitroxyl compounds are, in particular, 2,2,6,6-tetramethylpiperidine-N-oxyl (TEMPO) and derivatives (such as 4-hydroxy-, 4-acetoxy- and 4-acetamido-TEMPO) and analogous oxazolidine and pyrrolidine compounds. These can be used in catalytic amounts, for example 0.1-5% (mol/mol) with respect to the anticipated amount of polysaccharide (in monosaccharide equivalents). In this reaction the amount of re-oxidising agent (such as hypochlorite or oxygen) determines the final degree of oxidation of the product. Re-oxidation with hypochlorite is relatively simple to carry out. The somewhat more selective hypobromite can optionally be used as the actual re-oxidising agent by adding a catalytic amount of bromide, which is converted in situ into hypobromite by hypochlorite and is re-formed during the oxidation. It is also possible to oxidise the nitroxyl compound in advance, for example with oxygen or hydrogen peroxide and oxidative enzyme, to give a nitrosonium compound, which is added in this form in the desired amount to the biological material and is regenerated afterwards.

With the process according to the invention the polysaccharide can, if desired, be only partially oxidised, for example 3-30% oxidised, if the polysaccharide is intended for use in medicaments or foods. If desired for the intended application, the oxidation can also be carried out more extensively. For example 30-90% of the anhydroglycose units present can be oxidised. This more extensive oxidation is of importance especially for applications in which anionic or other functional groups are desired, such as in emulsifiers, binders, thickeners and the like. Moreover it was found that the separation of the oxidised polysaccharides is further facilitated at such higher degrees of oxidation. Particularly preferentially a 50-85% oxidation is carried out. What is meant by, for example, 30% oxidation is that the oxidation is carried out with an amount of re-oxidising agent such that when the re-oxidising agent is fully utilised a hydroxymethyl group is converted to a carboxyl group in 30% of the monomer units of the polysaccharide. In the case of hypochlorite as re-oxidising agent this therefore signifies 0.60 mol hypochlorite per mol monomer (anhydromonose), in accordance with the equation:

R—CH₂OH+2ClO⁻→R—COOH+2Cl⁻+H₂O

where R is the dehydroxymethylated residue of an anhydromonose unit. At higher degrees of oxidation it is possible, if desired, to use excess oxidising agent (re-oxidising agent), for example 2 or more mol re-oxidising agent (such as hypochlorite) per mol anhydromonose units.

Following oxidation to the desired degree, the oxidised polysaccharide can easily be isolated, for example by separating the reaction mixture into a soluble fraction, which contains the oxidised polysaccharide together with salts and other components that can easily be separated off, and an insoluble fraction, which contains mainly proteins and other biological material that is not desired for the application of the polysaccharide. With or without prior separation into a water-insoluble fraction and a water-soluble fraction, the oxidised polysaccharide can be precipitated by means of a non-solvent, such as ethanol or a higher alcohol. If desired, separation into water-soluble matter (the oxidised polysaccharides) and water-insoluble matter (usually protein-like material) can then be carried out. The carboxylic acid content (uronic acid) in the polysaccharide product can be determined in a known manner, for example by the method of Blumenkrantz et al. (Anal. Biochem. (1973) 54, 484), in which the product is hydrolysed with boric acid (0.0125 M) in concentrated sulphuric acid and 3-hydroxybiphenyl is then added and the extinction is measured at 520 nm.

The process according to the invention is not only suitable for the isolation of glucans from cell walls of yeasts, moulds, bacteria and other microorganisms, but also for the isolation of similar glucans or other polysaccharides from other biological material in which the polysaccharides are present together with protein material and other components that are difficult to separate. Examples of these are grasses, sugar beet residues, beet pulp, cereal fibres and other cereal residues (arabans, arabinoxylans and arabinogalactans), brewer's grains, plant cell walls and other vegetable residues (cellulose and hemicellulose). The great advantage of the process according to the invention is that no or little pre-separation of other biological components from the starting material is required.

However, it is necessary that the polysaccharides to be solubilised and/or to be isolated possess primary hydroxyl groups, as in 1,2-, 1,3- and 1,4-linked polyhexoaldo-pyranosides, 2,1- and 2,6-linked polyhexoketofuranosides, 1,2- and 1,3-linked polypentoketofuranosides and the like.

If desired, it is possible, as a supplement to a partial oxidation of primary hydroxyl groups, for a partial oxidation of the polysaccharides on other hydroxyl groups also to take place, such as by means of 2,3 oxidation in the case of (arabino)xylans and (arabino)galactans and other polysaccharides which contain —CHOH—CHOH— units, this unit being converted into two aldehyde groups and/or carboxyl groups. This oxidation can be carried out with, for example, hypochlorite, or periodate and chlorite as is known per se for the oxidation of polysaccharides. In this case the oxidation is preferably carried out on only 1-10% of the available anhydroglycose units to prevent excessive chain shortening and an excessive change in the spatial structure of the polysaccharide. It is also possible to carry out further derivative formation, such as esterification, etherification (for example hydroxyalkylation with ethylene oxide or propylene oxide or carboxymethylation with chloroacetic acid), crosslinking (with, for example, epichlorohydrin or dialdehydes or by intermolecular esterification) and other modifications known per se.

The invention not only relates to the process for the oxidation of the polysaccharides in the biological raw material but also to the products obtainable in this way, in particular β-1,3-glucurans. The uronic acid content of these products is in general 3-90%, more particularly 3-30%, 30-50% or 50-90%, partly dependant on the intended application. The products are virtually free from ketone, aldehyde and acid functional groups in positions other than the primary position (6-position).

The oxidised polysaccharides, in particular β-1,3-glucans, according to the invention can be used as health-promoting agents or medicinal excipients, in particular as immunity-promoting agents. They can also be used as a food component, either because of the calorific value, for example in animal feeds, or because of the value as dietary fibre or as a prebiotic in foods or nutraceutics for humans or other mammals or animals. For such applications amounts of, for example, 10 mg to 2 g per kg body weight, in particular 50 mg-1 g per kg, can be administered.

In particular the oxidised polysaccharides can be used as binders, absorbents, wetting agents for cosmetics or personal hygiene, thickeners and emulsifiers for foods, but also in paints, inks and the like, textile auxiliaries, metal-complexing agents, suspension agents in detergents, adhesives, protective colloids, pharmaceutical excipients and the like. In general the products according to the invention can be used where carboxymethylcellulose or other carboxymethylglucans are used according to the state of the art. For these applications they can be used as such, mixed with carriers or fillers, optionally in aqueous solution and optionally in combination with other active ingredients, in preparations, in amounts of, for example, 0.1-500 g, in particular 1-100 g per kg preparation. They can be used in these preparations in the acid form or in the form of a suitable salt, for example a salt with sodium, potassium, magnesium, calcium, zinc, ammonium and the like, or in the form of an ester.

The protein material from the biological raw material can frequently also be usefully used. When the polysaccharides have been separated off, the residual material can be used as protein material, after further purification if required. In the case of glycoproteins, such as the mannoproteins that are present in the yeast cell walls, these can also be partially oxidised and solubilised using the process according to the invention and optionally isolated from the polysaccharides by fractionation.

### EXAMPLE 1

Yeast flakes (20 g, 123.5 mmol anhydroglucose units, AGU) were stirred for 1 hour in water (200 ml) at pH 11. The pH was then adjusted to 10 and TEMPO (600 mg, 3.84 mmol, dissolved in 60 ml water) and NaBr (100 mg, 0.97 mmol) were added. A solution of HOCl (123.5 mmol) was added to the mixture using a metering pump. With the aid of a pH-stat the pH was kept constant by adding 0.5 M NaOH. The reaction was stopped after 2 hours. The reaction mixture was added to 100% ethanol. The reacted carbohydrates were filtered off and rinsed with ethanol. After filtration, the precipitate was dried. The dried product was taken up in water and centrifuged for 30 minutes at 10,000 rpm. The supernatant liquor was freeze-dried.

The product (17 g) contains 54% uronic acids (Blumenkrantz) and 3% protein (Gerhardt). The average molecular weight is 80,000 (HPLC).

### EXAMPLE 2

Yeast flakes (40 g, 246.9 mmol AGU were stirred for 1 hour in water (400 ml) at pH 11. The pH was then adjusted to 10 and TEMPO (1.2 g, 7.68 mmol, dissolved in 120 ml water) and NaBr (200 mg, 1.94 mmol) were added. A solution of HOCl (50 mmol) was added to the mixture using a metering pump. With the aid of a pH-stat the pH was kept constant by adding 0.5 M NaOH. The reaction was stopped after 1.5 hours. The reaction mixture was added to 100% ethanol. The reacted carbohydrates were filtered off and rinsed with ethanol and dried. The dried product was taken up in water and centrifuged for 30 minutes at 10,000 rpm. The supernatant liquor was freeze-dried.

The product (10 g) contains 5.5% uronic acids (Blumenkrantz) and 12.5% protein (Gerhardt). The average molecular weight is 50,000 (HPLC). The precipitate (after centrifuging) contains 3.5% uronic acids (Blumenkrantz) and 18% protein (Gerhardt). The average molecular weight is 42,000 (HPLC). The moisture content is 73%.

### EXAMPLE 3

The precipitate obtained after centrifuging from Example 2 (40 g, 67.4 mmol AGU) was adjusted to pH 10. TEMPO (500 mg, 3.2 mmol, dissolved in 80 ml water) and NaBr (100 mg, 0.98 mmol) were then added. A solution of HOCl (75 mmol) was added to the mixture. The pH was kept constant by adding 0.5 M NaOH. The reaction was stopped after 1 hour. The reaction mixture was added to 100% ethanol. The reacted carbohydrates were filtered off and rinsed with ethanol. After filtration, the precipitate was dried. The product (10 g) contains 50% uronic acids (Blumenkrantz).

### EXAMPLE 4

Example 2 was repeated, except that 85 mmol HOCl was added, that the solution was cooled to below 10° C. during the reaction and that the reaction was stopped after 2 hours. After filtration, the precipitate was dried.

The product (33 g) contains 9.3% uronic acids (Blumenkrantz). The average molecular weight is 51,000 (HPLC).

### EXAMPLE 5

Example 2 was repeated, except that 255 mmol HOCl was added in 7 portions (25-55 ml), that the solution was cooled to below 30° C. and that the reaction was stopped after 0.5 hour with 1% H₂O₂. After filtration, the precipitate was dried.

The product (36 g) contains 48% uronic acids (Blumenkrantz). The average molecular weight is 100,000 (HPLC). After the product had been fractionated using a P6 column it was found that the carbohydrate and protein fractions could not be separated.

### EXAMPLE 6

Example 2 was repeated, except that 255 mmol HOCl was added in 7 portions (25-55 ml), that the solution was cooled to below 30° C. and that the reaction was stopped after 0.5 hour with 1% H₂O₂. Before adding to ethanol, the reaction mixture was separated overnight into a precipitate and supernatant liquor. The liquor, which was not completely clear, was added to 100% ethanol. The reacted carbohydrates were filtered off. The precipitate was rinsed with ethanol and dried after filtration. The cloudy filtrate was evaporated and dried.

The product contains 81% uronic acids (Blumenkrantz) and 0.07% protein (Coomassie). The average molecular weight is 70,000 (HPLC). No large molecules were found in the cloudy filtrate.

### EXAMPLE 7

### Oxidation of Inactivated Dry Yeast with Laccase/Tempo

Inactivated dry yeast (10 g) and TEMPO (2.5 g) were taken up in 1 litre 20 mM succinate buffer, pH 5.5, and brought to 38° C. The reaction vessel was stirred and oxygen was bubbled through it. The reaction was started by adding 60 Units laccase (Trametes versicolor laccase, Wacker Chemie; TEMPO Units). During the reaction, which had a total duration of 6 hours, 20 Units laccase were added every hour and the pH was kept constant using a pH-stat. After completion of the reaction, the product was centrifuged and the dry weight of the supernatant liquor, the water-soluble fraction, was determined. This was found to be 3.1 g, corresponding to 31% of the starting material.

1-15. canceled

16. A process for the solubilisation and/or isolation of polysaccharides from a biological raw material that contains other biological materials in addition to the polysaccharides, characterised in that the raw material is treated with an oxidising agent that leads to selective oxidation of primary hydroxyl groups in the glucan.

17. A process according to claim 16, wherein the oxidising agent comprises a catalytic amount of a nitroxyl compound.

18. A process according to claim 16, wherein the oxidising agent comprises a hypohalite.

19. A process according to claim 16, wherein the oxidising agent comprises a peroxidase, a laccase or a polyphenol oxidase.

20. A process according to claim 16, wherein an amount of oxidising agent is used such that 30-90 hydroxyl groups per 100 anhydroglycose units can be oxidised to carboxyl groups.

21. A process according to claim 16, wherein the biological raw material also contains a protein material.

22. A process according to claim 16, wherein no prior separation between the polysaccharides and other biological material is carried out.

23. A process according to claim 16, wherein, following the oxidation, the treated polysaccharides are separated from other biological material, in particular proteins, by dissolving in an aqueous medium.

24. A process according to claim 16, wherein the polysaccharides comprise β-glucans, a part of the anhydroglucose units of which are linked via 1,3-bonds.

25. A process according to claim 24, wherein yeast cell residues are used as the source of the polysaccharides.

26. A process according to claim 16, wherein beet pulp or cereal residues are used as the source of the polysaccharides.

27. Oxidised β-glucan, at least a part of the anhydroglucose units of which are linked via 1,3 bonds, and in which 30-90 hydroxyl groups per 100 anhydroglucose units have been oxidised to carboxyl groups.

28. Oxidised β-glucan according to claim 27, having a chain length of 10-3000 anhydroglucose units.

29. Oxidised β-glucan according to claim 28, having a chain length of 20-1000 anhydroglucose units.

30. Oxidised polysaccharide containing anhydroarabinose units, in which 3-90 hydroxyl groups per 100 anhydroarabinose units have been oxidised to carboxyl groups.

31. A cosmetic or personal hygiene composition comprising an effective amount of an oxidised polysaccharide according to claim 27, as a wetting agent.

32. A cosmetic or personal hygiene composition comprising an effective amount of an oxidised polysaccharide according to claim 30, as a wetting agent.

33. A cosmetic or personal hygiene composition comprising an effective amount of an oxidised polysaccharide prepared according to claim 16, as a wetting agent.

34. A composition comprising an effective amount of an oxidised polysaccharide according to claim 27, as a binder, an emulsifier, or a thickener.

35. A composition comprising an effective amount of an oxidised polysaccharide according to claim 30, as a binder, an emulsifier, or a thickener.

36. A composition comprising an effective amount of an oxidised polysaccharide prepared according to claim 16, as a binder, an emulsifier, or a thickener.
